(19) United States
(12) Patent Application Publication
(10) Pub. No.: US 2025/0204801 A1
(43) Pub. Date: Jun. 26, 2025

(54) PERFUSION MONITORING

(71) Applicant: KONINKLIJKE PHILIPS N.V., EINDHOVEN (NL)

(72) Inventors: Tobias WISSEL, Lübeck (DE); Sven KROENKE-HILLE, HAMBURG (DE); Gerhardus Wilhelmus LUCASSEN, EINDHOVEN (NL); Rieko VERHAGEN, VUGHT (NL); Marc Godfriedus Marie NOTTEN, ELSLOO (NL)

(21) Appl. No.: 18/850,262

(22) PCT Filed: Mar. 27, 2023
(86) PCT No.: PCT/EP2023/057776

§ 371 Date: Sep. 24, 2024

(30) Foreign Application Priority Data
Mar. 31, 2022 (EP) 22165791.9

(51) Int. Cl. A61B 5/026; A61B 5/00; A61B 6/50; G06T 7/00; G06T 7/33; G06T 11/00; G06V 10/25; G16H 30/40

(52) U.S. Cl. CPC A61B 5/0261; A61B 5/7485; G06T 7/0016; G06T 7/344; G06T 11/00; G06V 10/25; G16H 30/40; A61B 5/0263; A61B 6/507; A61B 2576/02; G06T 2207/10088; G06T 2207/10116; G06T 2207/30104; G06V 2201/03

(57) ABSTRACT

The present disclosure relates to perfusion monitoring. In order to provide facilitated peripheral perfusion monitoring, a device (10) for monitoring peripheral perfusion is provided that comprises a data input (12), a data processor (14) and an output interface (16). The data input is configured to provide angiographic image data comprising information about macrovascular blood flow in an area of interest of a subject. The angiographic image data comprises first image data relating to a first point in time, and second image data relating to a second point in time. The data processor is configured to compare the first image data and the second image data to identify, within the area of interest of the subject, a vascular region of interest for macrovascular flow. The data processor is also configured to determine at least one tissue portion of interest for microvascular perfusion based on the identified vascular region of interest and based on feeding information assigned to the identified vascular region of interest. The data processor is further configured to determine a surface portion for assessing microvascular perfusion in the at least one tissue portion of interest with optical perfusion imaging. The data processor is furthermore configured to allocate the surface portion on an outer surface of the subject. The output interface is configured to provide a surface portion identifier based on the allocated surface portion.

### FIELD OF THE INVENTION

The present invention relates to perfusion monitoring and to microvascular perfusion monitoring for assisting interventions. Disclosed are methods, devices and systems for such purposes including monitoring peripheral perfusion or the assistance therein.

### BACKGROUND OF THE INVENTION

Atherosclerosis in the peripheral arteries is a chronic and slowly developing condition causing narrowing of the arteries. Depending on the degree of narrowing, various symptoms may occur with patients developing acute events associated with thrombosis and/or embolism and occlusion of a major artery. Narrowing of the arteries leads to peripheral artery disease (PAD). Narrowing of arteries can also result in coronary artery disease. Current clinical methods to diagnose PAD are based on blood pressure measurements like Ankle Brachial Index (ABI), Toe Brachial Index (TBI) and Toe Pressure (TP), which measure ratios of blood pressures at ankle or toe and the arm. Further, transcutaneous oxygen tension (TcpO2) measurements are performed to get oxygen tension values after heating tissue, but these measurements are cumbersome. Furthermore, to look at the global perfusion, digital subtraction angiography (DSA) is used which provides a 2D projection of the vascular system where injected contrast fluid flows through the artery and veins. Stenoses due to calcified arteries or compressed veins, thrombosis or a lack of perfusion at the lower limbs as their consequence can be readily visualized, but sometimes at the cost of radiation and contrast. EP 3808275 A1 provides perfusion angiography combined with photoplethysmography imaging for peripheral vascular disease assessment. However, concurrent acquisition provides a complex workflow.

### SUMMARY OF THE INVENTION

There may thus be a need to provide facilitated peripheral perfusion monitoring.

The object of the present invention is solved by the subject-matter of the claims. It should be noted that the following described aspects of the apply for the devices, systems and methods disclosed herein.

According to the present disclosure, a device as claimed in claim 1 is provided. Such device may be a device for microvascular perfusion monitoring or for the assistance therein. Alternatively, or additionally, such device may be for monitoring peripheral perfusion or for assisting therein. The device comprises a data input, a data processor, and an output interface. The data input and output interface are communicatively coupled to the data processor to perform their functions. The data input is configured to provide (to the data processor) flow data comprising information about macrovascular blood flow in an area of interest of a subject. In some examples the flow data comprises or consists of angiographic image data which image data comprise first image data relating to a first point in time, and second image data relating to a second point in time. The data processor is configured to identify a vascular region of interest for macrovascular flow based on or using the flow data. When the flow data comprises or consists of the angiographic image data, the data processor can be configured to compare the first image data and the second image data to identify, within the area of interest of the subject, the vascular region of interest for macrovascular flow. The data processor is also configured to determine at least one tissue portion of interest for microvascular perfusion, wherein such determination is based on the identified vascular region of interest and on feeding information assigned to the identified vascular region of interest. In other words, the data processor is configured to use the feeding information and the identified vascular region to identify the at least one tissue portion. The data processor is further configured to determine a surface portion for assessing microvascular perfusion with optical perfusion imaging. The surface portion is determined to be part of or within the at least one tissue portion of interest the data processor is configured to allocate the surface portion on an outer surface of the subject or on an outer surface of an organ of the subject. The output interface is configured to generate or provide a surface portion identifier based on the allocated surface portion.

As an effect, the present disclosure enables to directly link occluded, partially occluded, or re-opened microvasculature as an immediate effect of an intervention, such as for example observed with angiographic imaging (e.g. X-ray, Magnetic Resonance (MR) or other), to the optical measurements of perfusion at a relevant symptomatic site given by the allocated surface portion. Angiography as basis of perfusion on the macrovascular level thus provides an improved guidance of optical measurements on the microvascular level and a confirmation of specific angiographic observations at the skin or other organ surface may be performed. Alternatively, or additionally, optical perfusion measurements at a surface of a subject may be aid check of an effect of an intervention aimed at reopening of a macrovasculature.

According to an example, the data processor is configured to generate a perfusion hypothesis based on the flow data. For example, when the flow data comprise angiographic image data including the first and second image data, the hypothesis can be based on the comparing of the first image data and the second image data and the identification of the vascular region of interest for macrovascular flow.

The data processor is configured to generate, as the identifier, instructions for optical perfusion measurement (e.g optical perfusion imaging) for at least one of the group consisting of quantifying and validating of the perfusion hypothesis.

According to an example, the data input is configured to provide feeding information comprising at least one of the group consisting of locational information and semantic information for the vascular structure, preferably of at least one of the group consisting of the first image data and the second image data. The data processor is configured to determine the at least one tissue portion of interest for microvascular perfusion using the semantic information. Additionally, or alternatively, the data processor is configured to allocate the surface portion using the locational information.

In some examples the feeding information is provided by the data input or a further data input. In some examples the feeding information is stored in a memory accessible to the data processor.

According to an example, the angiographic image data comprises spatial anatomical information comprising a plurality of vessel segments. For the identification of the vascular region of interest for macrovascular flow, the data processor is configured to identify at least one of the plurality of vessel segments. The feeding information comprises segments of tissue portions that are assigned to vessel segments of the plurality of vessel segments as being supplied by these vessel segments, or drained by these vessel segments Preferably, they are assigned as being supplied by these vessel segments.

According to an example, the feeding information comprises a feeding territory model. The data processor is configured to spatially register the angiographic image data to the feeding territory model. The data processor is configured to register the feeding territory model to the subject.

According to an example, the output interface is configured to provide optical perfusion image data. The optical perfusion image data comprises at least one 2D or 3D optical perfusion image of the surface of the subject. The data processor is configured to generate an indicator for the allocated surface portion. The data processor is also configured to overlay or integrate the indicator to the at least one 2D or 3D optical perfusion image to provide a location augmented optical perfusion image. The output interface is configured to provide the location augmented optical perfusion image for a perfusion assessment in the image portion defined by the indicator.

According to the present disclosure, also a system for monitoring microvascular and/or peripheral perfusion or the assistance therein is provided. The system comprises a device as defined according to any of the preceding examples. The system further comprises an optical perfusion measurement arrangement/system for measurement of microvascular perfusion within the allocated surface of the subject. The optical perfusion arrangement is configured to conduct for example optical perfusion imaging of the allocated surface portion based on the surface portion identifier. In some examples the system comprises a source system as described herein for storing and/or generating the flow data, which system is connectable to the data input for communicating the flow data to the data input. In some examples such source system comprises an angiographic imaging arrangement/system for angiography of a region of interest of a subject. Such angiographic system may comprise one or more of the group consisting of: X-ray, Magnetic resonance and other imaging systems. The X-ray, MR or other angiographic imaging system is configured to provide the angiographic image data.

According to an example, a spatial correspondence arrangement is provided that is configured to provide relative spatial correspondence data of the X-ray or other imaging arrangement/system and the optical perfusion arrangement/system.

In an example, a location detection system is provided to detect current positions and locations of both arrangements/systems. For example, the location detection is based on electromagnetic markers. In another example, optical detection of marker locations is provided.

According to the present disclosure, also a method is provided. The method may be for microvascular and/or peripheral perfusion imaging or the assistance thereof is provided. The method comprises the following steps:
Using flow data, for example angiographic image data, comprising information about macrovascular blood flow in an area of interest of a subject. The angiographic image data comprises first image data relating to a first point in time and second image data relating to a second point in time.
Comparing the first image data and the second image data to identify, within the area of interest of the subject, a vascular region of interest for macrovascular flow.
Determining at least one tissue portion of interest for microvascular perfusion based on the identified vascular region of interest and based on feeding information assigned to the identified vascular region of interest.
Determining a surface portion for assessing microvascular perfusion in the at least one tissue portion of interest with optical perfusion imaging.
Allocating the surface portion on an outer surface of the subject or organ thereof; and
Providing a surface portion identifier based on the allocated surface portion.

The method may be computer implemented. In such case the device may include a memory storing instructions which when executed by the data processor or a computer including the data processor causes the device or system to perform any steps of the method.

According to an aspect, the current disclosure proposes to locate and spatially link a (macrovascular) perfusion signal of an angiography (or lack thereof) to a relevant surface area for (i) choosing the most relevant field-of-view for an optical perfusion measurement, and (ii) for validating a re-perfusion hypothesis as expected from the perfusion angiography against data from the optical surface perfusion measurement such as an optical perfusion map.

According to an aspect, two modalities are combined in a stereo-imaging configuration for transferring information from angiographic to optical surface measurements for obtaining sophisticated insights into perfusion properties of limbs. A macrovascular perfusion signal of the angiography (or a lack thereof) is located and spatially linked to the relevant surface area to (i) choose the most relevant field-of-view for the optical measurement, and, as an option, (ii) to validate re-perfusion hypotheses as expected from perfusion angiography against data from the optical surface perfusion map. As an example, optical perfusion monitoring such as iPPG, MSI, SLI or combinations thereof measure the blood content, flow or oxygenation directly where necrotic wounds and other symptoms actually occur—within a few millimeters of the skin surface.

In an example, it is provided to measure peripheral perfusion and/or tissue oxygenation at the skin surface using optical illumination with e.g. contact probes, such as laser doppler imaging, speckle contrast imaging, or with non-contact imaging methods using a camera, e.g. imaging photoplethysmography (iPPG), laser speckle imaging (LSI) or multi/hyperspectral imaging (MSI/HSI). These techniques give insight of the microvascular perfusion and oxygenation which can then be provided to decide on e.g. treatment, predict wound healing and outcome.

In an example, a system comprises an optical perfusion measurement device, e.g. hyper- or multispectral iPPG with structured light, and a post-processing unit or module that maps the measured signal, e.g. diffuse reflectance spectrum, to hemodynamic parameters, e.g. oxygen saturation. The system further comprises an angiography imaging system, e.g. a C-arm X-ray imaging system, that may be provided in a stereo configuration with respect to an optical imaging system, with known or measurable relative positioning/orientation, e.g. by using senor hardware or by pose-estimation using the acquired images. The system furthermore comprises an analysis unit or module with a user interface, which establishes spatial correspondence between the modalities and transfers locational information and also hypotheses about the perfusion status from angiography to the optical surface domain for an advanced analysis of perfusion properties. The present disclosure leverages the complementary properties of optical and angiographic perfusion measurements and overcomes up to a certain degree the limitations of one modality by using the other modality.

As an example, when referring to a combination of photoplethysmogram imaging (iPPG), multi-spectral imaging (MSI) and structured light imaging (SLI), chemical sensitivity (oxygenation etc.) is provided. A penetration depth of approximately 2 mm is possible, which has to be seen though in addition to a depth of larger than 10 cm of the angiography imaging. Further, a typical view of imaging may be provided as sole/en-face, i.e. surface imaging, in addition to angiography's usually lateral volume projection. The optical imaging is providing a high sensitivity to microvascular perfusion, whereas angiography is providing a high sensitivity to macrovascular perfusion. The optical imaging enables to observe blood perfusion related parameters in absolute units, such as for oxygen saturation, or SLI can facilitate blood-volume-content measurements. Since optical imaging is a purely optical measurement, the subject is not exposed to any additional radiation dose. As a further complementation, optical imaging may be provided with a frame rate of approximately 24 frames per second, in addition to a frame rate of e.g. three frames per second of angiography imaging.

As an aspect, both imaging modalities provide relevant and complementary pieces of information about the effect of an intervention on the perfusion status. The present disclosure provides the explicit connection of the information.

As an example, DSA sequences are acquired, e.g. in a repetitive manner, to evaluate re-perfusion in the macrovascular tree in the foot. Seeing increases in perfusion in previously ischemic regions gives rise to the hypothesis that tissue perfusion and oxygenation in the surface wound area are also positively affected. This hypothesis is then quantified and used for a tailored optical validation. In an example, the present disclosure identifies perfusion deficits and regions of interest in the angiography and uses this for guiding the optical measurement. Explicit hypotheses on how an angiographically observed change in perfusion will affect tissue perfusion at the skin surface, are formulated and brought into the optical measurement for validation.

These and other aspects of the present disclosure will become apparent from and be elucidated with reference to the embodiments described hereinafter.

### BRIEF DESCRIPTION OF THE DRAWINGS

Exemplary embodiments of the disclosure will be described in the following with reference to the following drawings:

FIG. 1 schematically shows an example of a device for monitoring peripheral perfusion.

FIG. 2 shows an example of a system for monitoring peripheral perfusion comprising an example of the device for monitoring peripheral perfusion of FIG. 1.

FIG. 3 shows steps of an example of a method for peripheral perfusion imaging.

FIG. 4 illustrates an example of a workflow for peripheral perfusion imaging.

FIG. 5 illustrates a further example of a workflow for peripheral perfusion imaging.

### DETAILED DESCRIPTION OF EMBODIMENTS

Certain embodiments will now be described in greater details with reference to the accompanying drawings. In the following description, like drawing reference numerals are used for like elements, even in different drawings. The matters defined in the description, such as detailed construction and elements, are provided to assist in a comprehensive understanding of the exemplary embodiments. Also, well-known functions or constructions are not described in detail since they would obscure the embodiments with unnecessary detail. Moreover, expressions such as “at least one of”, when preceding a list of elements, modify the entire list of elements and do not modify the individual elements of the list.

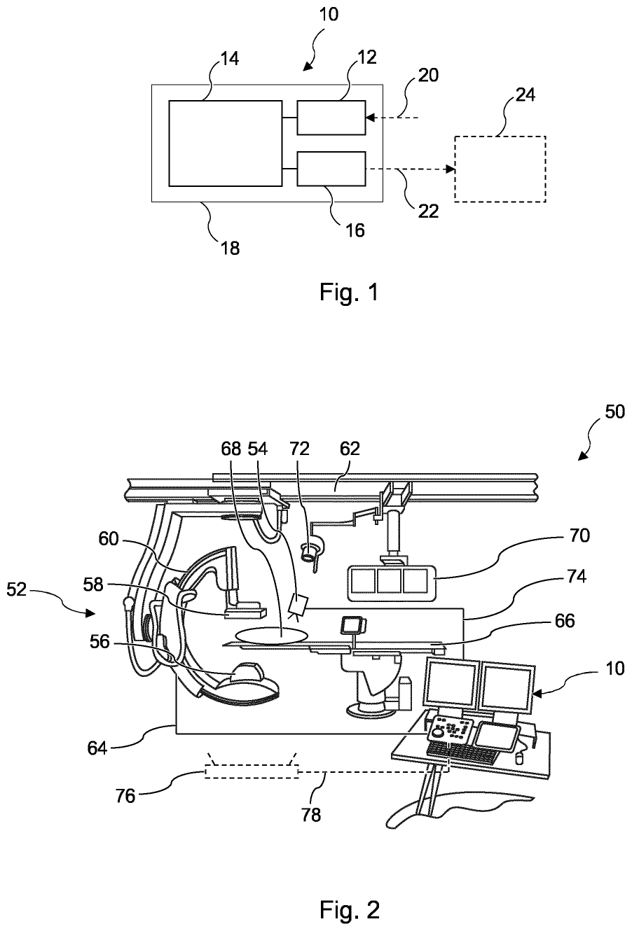

FIG. 1 shows an example of a device 10 for assisting monitoring of microvascular perfusion. In some examples microvascular perfusion may comprise or consist of peripheral perfusion. The device 10 comprises a data input 12, a data processor 14 and an output interface 16.

The data input 12 is configured to provide flow data comprising information about macrovascular blood flow in an area of interest of a subject. For example, it may comprise data representative of parts of the macrovasculature with reduced or impaired macrovascular blood flow as compared to normal blood flow. Such flow data may have been obtained from angiography measurements. In some examples such flow data comprise or consist of angiographic image data. The angiographic image data comprises first image data relating to a first point in time, and second image data relating to a second point in time. The angiographic image data thus include time sequential image data from which a blood flow in the macrovasculature may be determined.

The data processor 14 is configured to use the flow data to identify a vascular region of interest within the area of interest of the subject. In some examples the data processor 14 is configured to receive the first and second image data and compare the first image data and the second image data to identify the vascular region of interest for macrovascular flow from the comparison

The data processor 14 is also configured to determine at least one tissue portion of interest for microvascular perfusion based on the identified vascular region of interest and based on feeding information assigned to the identified vascular region of interest. The data processor 14 is further configured to determine a surface portion for assessing microvascular perfusion in the at least one tissue portion of interest with optical perfusion imaging. The data processor 14 is furthermore configured to allocate the surface portion on an outer surface of the subject or of an organ of the subject. The outer surface of the subject may thus include any surface forming a boundary of the subject with the environment (e.g. surrounding atmosphere) for example during an intervention.

The output interface 16 is configured to provide a surface portion identifier based on the allocated surface portion. Such identifier may comprise data indicative of at least a part of the allocated surface. Such identifier may be used for provision to a user and/or used for controlling an optical perfusion measurement arrangement/system. The data input 12, the data processor 14 and the output interface 16 can be provided in an integrated manner, as indicated by a first frame 18. In another option, the data input 12, the data processor 14 and the output interface 16 are provided as separate parts or arrangements.

The data input and output interface are communicatively coupled to the data processor. This ensures that the data processor can receive the relevant flow data (e.g. supply data) from the data input for processing and communicate the relevant output data to the output interface. It will be clear that the data input is configured to be connectable to an input system or input device such that it can receive the relevant flow data from such input system or input device, and it will be also clear that the output interface is configured to be connectable to an output system or output device that may receive or obtain the result data form the output interface for further use. For example, the input system or input device may be an angiographic imaging system or repository storing angiographic imaging data. Further examples will be described herein below. Examples of output systems or output devices can include user interfaces such as display devices for displaying images. Alternatively, or additionally, the output system may comprise an optical perfusion imaging system. Other examples will be described herein. A first hashed arrow 20 indicates the provision of the above mentioned supply data (e.g. the flow data), and a second hashed arrow 22 indicates the generated (result) data e.g. in form of the surface portion identifier.

The surface portion identifier may be provided on a display 24 indicated in FIG. 1 with a second frame shown in hashed lines.

The term “area of interest of a subject” relates to a body part or portion of the subject, in which changes in macrovascular blood flow can be expected, e.g., due to an interventional procedure or due to an existing obstruction of blood flow. The term macrovascular blood flow relates to blood flow in vessels that are visible with angiographic imaging techniques such as, for example, X-ray, Magnetic resonance (MR) or other imaging techniques. In some examples such X-ray or MRI techniques are used in combination with contrast injection in vessels. Often X-ray imaging with contrast injected X-ray images of the vessels is used.

The term “vascular region of interest” relates to a part or portion of the vascular structure of the subject, in which changes in the macrovascular blood flow are present or are detected, for example, by the comparison of the image data relating to the different points in time.

The term “tissue portion of interest” relates to a part or portion of the tissue structure of the subject, in which changes in the macrovascular blood flow might most probably cause changes in the microvascular blood flow (perfusion) based on the feeding information for the respective identified vascular region of interest.

The term “microvascular blood flow” relates to blood flow in the smaller parts of the vascular structure (often referred to as the microvasculature). In some examples such microvasculature may be the peripheral vascular structure. The microvascular blood flow also relates to blood perfusion in tissue. Usually, microvascular perfusion is hardly visible in common angiographic images such as contrast injected X-ray images or contrast injected MR images of the vessels.

The term “surface portion” relates to a part or portion of the (outer, e.g. visible) surface of the respective tissue portion. The surface portion can also be referred to as surface area. Such surface portion may be part of the boundary (internal and/or external) of an organ of the subject. One example would be a part of the skin of the subject or part of a bowel of a subject during intervention.

The term “allocating the surface portion” relates to defining the surface portion. The surface portion identifier relates to any information that defines the allocated surface portion. The surface portion identifier may include for example coordinates, a delineation, an area, surface area or other method to identify or define the allocated surface portion as part of a coordinate system and/or of a representation of the outer surface of the subject or an organ thereof.

In an example, the surface portion is allocated on a representation of the surface of the subject or an organ thereof, e.g., on an image or model of the surface of the subject or organ thereof. The representation of the surface may take many forms. For example, an optical perfusion imaging is provided that captures a continuous stream of surface images and the indicator is overlaid on or integrated into a presentation of the surface images to identify the allocated surface portion. In another example the representation of the surface is based on an atlas model provided to the data processor or otherwise accessible to the data processor (e.g. stored in a memory). In such case the optical perfusion may be simplified in that it does not need to be imaging based. Thus, any other type of technique for measuring microvascular perfusion at the surface portion allocated may be used in such case. Examples such as simple optical perfusion measurement or electrical perfusion measurement are known in the art.

In an example, the surface portion is allocated on the actual surface of the subject or an organ thereof, e.g. directly on the surface, for example by an indicator projected onto the surface.

In an example, the allocated surface portion is provided as a preferred surface portion. The perfusion measurement arrangement or the perfusion imaging arrangement may thus be directed to the allocated surface as indicated for optical surface perfusion measurements to be conducted thereon.

The allocation is preferably done intra procedural, i.e. just before or during an ongoing medical intervention. This may aid in conducting the interventional procedure and possibly improve outcome and/or reduce intervention time. It may provide optical perfusion information that is linked to any blood flow problems observed during the intervention or even treated during the intervention and therewith an additional, microvascular perfusion based, check of diagnosis or of the effect of the intervention of the blood flow problem.

The term “data input” relates to providing or supplying data for data processing steps. The data input 12 can also be referred to as image data input. The data input can also be referred to as data supply, as image data supply, as image input, as input unit or simply as input. The term may indicate an activity of, or hardware for, receiving or obtaining the flow data 20 and providing such data to the data processor. A data input can be a single type of input but may also encompass several types of inputs possibly having several types of connectors to either the data processor or external devices. For instance different types may be used for different types of information communicated.

The term “data processor” relates to a processor or part of a processor system or processor arrangement that is provided to conduct the computing steps using the data supplied by the data input. The data processor 14 can also be referred to as data processing arrangement, as processor unit, processor circuit, or as processor. In an example, the data processor is data-connected to the data input and the output interface.

In an example, the data processor is provided as vascular flow determination engine that detects and/or tracks macrovascular blood flow in the angiographic images. In an example, a contrast agent tracking over time is technically not required, e.g. if someone chooses a CCN or thresholding of the DSA for this purpose.

The term “output interface” relates to an interface for providing the processed or computed data for further purposes. The output interface 16 can also be referred to as output or output unit. The output interface is configured to receive or obtain generated data or result data from the data processor and to make them available for further purposes when suitable devices or systems are connected to it. In an example, the output interface is data-connectable to a user interface such as for example display arrangement, display device, or display. Alternatively or additionally, the output interface is connectable to a repository (i.e. data storage) for storing results generated by the data processor. The output interface in some examples is connectable to an optical perfusion measurement arrangement to allow the latter to use the surface portion identifier.

The communication between the data input and the data processor and that of the output interface and the data processor may be provided by communications hardware buses and protocols for operating these as they are known in the art for electronic equipment, processors or computers. In some examples the data processor may include a memory such as D-RAM, S-RAM or the like or non-volatile memory such as flash, EEPROM etc. as known in the art for storing computer programs or instructions as defined herein below to cause the processor to perform steps described herein when executed by the processor. The feeding data may be stored on such memory but may also or alternatively be received or obtained form an external repository via the data input or a further data input that is configured to provide such data to the data processor. Data connection to the user interface may be local using cables or wireless connection, but may alternatively or also be remote e.g. via communications network. The data input and output interface may be configured to be connectable to a communications network such as LAN or WLAN or WWW or the like as known in the art.

In an example, the data input is data-connectable to an imaging source arrangement such as an angiographic imaging system for receiving the flow data. Exemplifying angiographic imaging systems include: X-ray- or Magnetic Resonance Imaging (MRI) systems. Other imaging source arrangements include a repository storing the flow data such as the angiographic image data. The repository may be standalone or part of an angiographic imaging system.

In a further example, in addition or alternatively, the data input is data-connectable or data connected to an optical camera providing the optical perfusion measurement such as e.g. the optical perfusion imaging of the subject. In an example, the data input is data-connected to an optical perfusion imaging source like a camera or other optical sensor system capable of providing the optical images. In an example, the data input is data-connectable to a data storage having stored the optical perfusion image data, which data storage may be standalone or part of the optical perfusion imaging source.

In an example the flow data include flow data of a pre-interventional state and intra-or post-interventional state, for example, the first image data relates to a pre-interventional state of a vessel to be treated, and the second image data relates to an intra- or post-interventional state. As an example, the intervention provides treatment of the vessel structure to improve blood flow at least in the vessel. In such case, an effect of treatment may be further assessed using the optical perfusion imaging data of the outer surface area identified by the system. An expected or hypothesized effect of the treatment as determined by the system for the outer surface may be compared with an actual measured effect. As another example the intervention provides injection of contrast agent to identify an occlusion of microvasculature to be treated during a further intervention to resolve the occlusion.

In an example, the surface portion is related to the determined at least one tissue portion of interest for microvascular perfusion.

In an example, the feeding information relates to the identified vascular region of interest. The feeding information may provide information on which perfused vessel (thus supplied with blood) supplies which tissue region. Alternatively, or additionally the feeding information may provide or include information on which perfused vessel (thus draining blood) drains which tissue region. The feeding information thus relates parts of the microvasculature to their most likely or defined blood sources in the macrovascular arteries and arterioles and/or to its most likely or defined blood drains in the macrovascular veins and venules. Preferably the feeding information relates to only the blood sources.

The feeding information is based on a model of the semantic affiliation, i.e. a model defining which vessel supplies or drains which tissue region. One approach is the identification of one or more regions in the model, for example in an angiosome model. Another or additional approach is the assumption of a spatial proximity. In some examples the model may be based on an atlas model. In other examples the model may be based on an actual vascular structure as imaged from the subject for example using known X-ray, MR, Ultrasound, or other imaging techniques. In some examples the model is based on both types.

In an example, a supply and/or drain vascular tree is provided for providing the feeding information.

FIG. 2 shows an example of a system 50 for monitoring peripheral perfusion or microvascular perfusion. The system 50 further comprises an example of the device 10 for assisting the monitoring of microvascular or peripheral perfusion according to one of the preceding examples. The system 50 optionally comprises an angiographic imaging system. In this example an X-ray imaging arrangement 52 for angiography of a region of interest of a subject is shown. The X-ray imaging system 52 is configured to provide the angiographic image data. The system 50 also comprises an optical perfusion arrangement 54 for measurement of perfusion within a surface of the subject. The optical perfusion arrangement 54 is configured to conduct optical perfusion imaging of the allocated surface portion based on the surface portion identifier. In alternative systems 50 the microvascular perfusion may be measured using different measurement techniques as indicated herein before.

The X-ray imaging arrangement 52 can also be referred to as X-ray imaging system or as angiography imaging system. In an example, the angiography imaging system is provided as e.g. a C-arm structure. As an option, the angiography imaging system comprises a stereo configuration with respect to the optical imaging system presented by the optical perfusion arrangement, for example with known or measurable relative positioning/orientation, by using e.g. sensor hardware or by pose-estimation using the acquired images.

The optical perfusion arrangement 54 can also be referred to as optical perfusion imaging system, optical perfusion measurement arrangement or system, or as optical perfusion measurement device. In an example, the optical perfusion arrangement is provided e.g. as hyper- or multispectral iPPG with structured light.

Still further, a post-processing is provided that maps the measured signal, e.g. diffuse reflectance spectrum, to hemodynamic parameters, e.g. oxygen saturation.

In an example, the optical perfusion arrangement 54 is configured to conduct optical perfusion imaging at predetermined points in time.

In another example, the optical perfusion arrangement 54 is configured to conduct optical perfusion imaging in a continuous manner as an image stream.

In FIG. 2, the X-ray imaging arrangement 52 is provided comprising an X-ray source 56 and an X-ray detector 58 attached to a C-arm structure 60 movably mounted to a ceiling support 62. The device 10 for monitoring peripheral perfusion is provided integrated within a console arrangement that may comprise user interfaces such as displays, keyboards, control panels, touch pads, mouse and the like. The console is shown in the right foreground in FIG. 2. The X-ray imaging arrangement 52 is data-connected to the device 10 for monitoring peripheral perfusion as indicated by a first data-connection line 64. Further, a subject support 66 is shown as an adjustable table to provide support for a subject 68. Further equipment like a monitor arrangement 70 and lighting 72 are also indicated. The optical perfusion arrangement 54 is data-connected to the device 10 for monitoring peripheral perfusion as indicated by a second data-connection line 64.

As an option, shown in FIG. 2 with hashed lines, a spatial correspondence arrangement 76 is provided that is configured to provide relative spatial correspondence data of the X-ray imaging arrangement 52 and the optical perfusion arrangement 54. The spatial correspondence arrangement 76 is data-connected to the device 10 for monitoring peripheral perfusion as indicated by a third data-connection line 78.

The data-connections may be provided wire-bound or wireless.

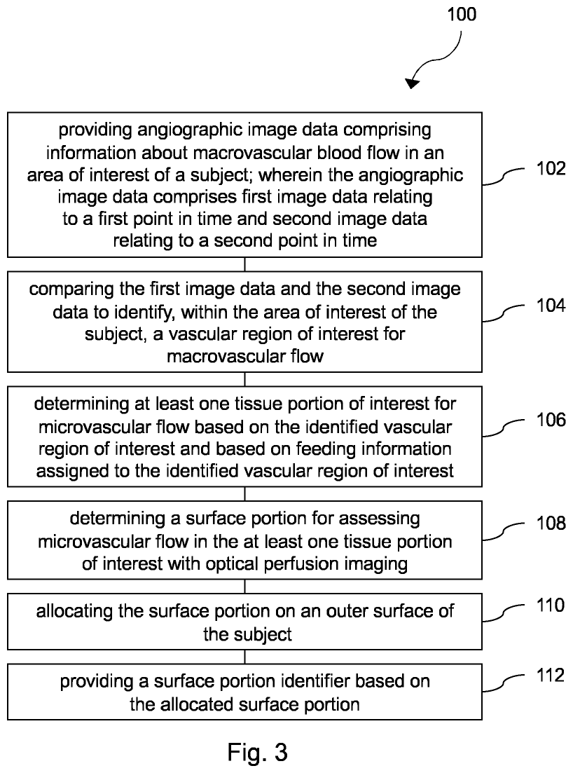

FIG. 3 shows steps of an example of a method 100 for peripheral perfusion imaging. The method 100 comprises the following steps:
In a first step 102, flow data are provided, for example in the form of angiographic image data, comprising information about macrovascular blood flow in an area of interest of a subject is provided. The angiographic image data comprises first image data relating to a first point in time and second image data relating to a second point in time.
In a second step 104, the first image data and the second image data are compared to identify, within the area of interest of the subject, a vascular region of interest for macrovascular flow. In some examples, the flow data represent flow characteristics pertaining to macrovascular regions. In such cases the vascular region of interest may be, for example, identified based on comparing the flow characteristics to threshold values that are considered to delineate normal from low blood flow. The vascular regions of interest in such cases would be identified as those macrovascular parts that have flow characteristics indicative of the low blood flow. In such cases the flow data do not need to have the angiographic image data and the method does not need to include the comparison step 14 to do the identification of the vascular region of interest from the image data.
In a third step 106, at least one tissue portion of interest for microvascular perfusion is determined based on the identified vascular region of interest and based on feeding information assigned to the identified vascular region of interest.
In a fourth step 108, a surface portion of the at least one tissue portion of interest is determined. The surface portion is for assessing microvascular perfusion with optical perfusion imaging. in the at least one tissue portion of interest with optical perfusion imaging.
In a fifth step 110, the surface portion is allocated on an outer surface of the subject or an outer surface of an organ of the subject. The allocated surface portion may be for example an outer surface portion of a bowel or of the skin of the subject.
In a sixth step 112, a surface portion identifier is provided based on the allocated surface portion.

Referring back to FIG. 1 and FIG. 2, in an example, provided as an option, the data processor 14 is configured to generate a perfusion hypothesis based on the comparing of the first image data and the second image data and the identification of the vascular region of interest for macrovascular flow. The data processor 14 is configured to generate, as the identifier, instructions for optical perfusion imaging for at least one of the group of quantifying and validating of the perfusion hypothesis.

In an example, the generated perfusion hypothesis is used to identify and define instructions for the optical perfusion imaging for the quantifying and/or validating of the perfusion hypothesis.

In an example, the optical perfusion imaging is performed based on the instructions for the optical perfusion imaging.

For example, the instructions comprise the most relevant field-of-view for the optical measurement.

In an example, the quantifying comprises determining absolute values and relations of the measured optical perfusion image data.

In another example, alternatively or in addition, the validating comprises validating the perfusion hypothesis as expected form perfusion angiography against data from the measured optical perfusion image data.

In an example, the allocated surface portion is used for comparing expected surface perfusion differences with measured surface perfusion differences.

As an example, a mismatch of hypothesis and measured perfusion is detected via absolute values of the measurements.

As another example, a mismatch of hypothesis and measured perfusion is detected via differences in values or via pre- and post-images.

In an example, provided as an option, the data input 12 is configured to provide at least one of the group of locational information and semantic information for the vascular structure of at least one of the group of the first image data and the second image data. Further, the data processor 14 is configured to determine the at least one tissue portion of interest for microvascular perfusion using the semantic information. As an additional or alternative option, the data processor 14 is configured to allocate the surface portion using the locational information.

In an example, the locational information is based on segmenting vessels in the angiographic image data. The segmenting can be provided manually by the user or by a segmentation algorithm.

The term locational information relates to data relating to a relative position of the vascular structure. The locational information thus provides the spatial context.

The term semantic information relates to data relating to an anatomical context of the particular part of the vascular structure, such as which sub-branches or which tissue regions are commonly supplied by the part of the vascular structure. The semantic information thus provides the functional context.

In an example, 2D angio images are provided with labels for vessel segments, thus providing 3D information over the generic model, for example. This may be provided in order to assess if vascular regions are supplied with blood flow.

In an example, provided as an option, the data processor 14 is configured to register the angiographic image data and the subject to a common spatial reference frame. Further, the data processor 14 is configured to allocate the surface portion based on the common spatial reference frame.

In an example, provided as an option, the angiographic image data comprises spatial anatomical information comprising a plurality of vessel segments. For the identification of the vascular region of interest for macrovascular flow, the data processor 14 is configured to identify at least one of the plurality of vessel segments. The feeding information comprises segments of tissue portions that are assigned to vessel segments of the plurality of vessel segments as being supplied by these vessel segments.

In an example, the angiographic image data comprises a number of angiographic projection images of the subject from at least two different directions such that the angiographic image data comprises is providing spatial information of the subject.

In an example, provided as an option, the feeding information comprises a feeding territory model. The data processor 14 is configured to spatially register the angiographic image data to the feeding territory model. The data processor 14 is also configured to register the feeding territory model to the subject.

In an example, an anatomical model is provided that is linked to the feeding territory model. The angiographic image data is registered to the anatomical model. In an option, also the optical perfusion image data is registered to the anatomical model.

In an example, provided as an option, the area of interest of the subject comprises the lower limbs. The at least one tissue portion of interest for microvascular perfusion comprises at least one foot region of the subject.

In an example, provided as an option, the output interface 16 is configured to provide location augmented optical perfusion image data. The location augmented optical perfusion image data comprises at least one 2D or 3D optical perfusion image of the surface of the subject. The data processor 14 is configured to overlay or integrate the surface portion identifier to the at least one 2D or 3D optical perfusion image to provide the location augmented optical perfusion image. The output interface 16 is configured to provide the location augmented optical perfusion image for a perfusion assessment in the image portion defined by the identifier.

The location augmented image provides information where measurements are relevant in an intuitive manner. The images may be displayed on a or the display connectable to the output interface.

In an example, provided as an option, for overlaying or integrating the surface portion identifier, the data processor 14 is configured to register the at least one 2D or 3D optical perfusion image with the angiographic image data.

In an example, for registering the at least one 2D or 3D optical perfusion image and the angiographic image data, a spatial relation between the two imaging modalities is determined.

In an example, the spatial relation is provided based on the angiographic image data.

In another example, a tracking arrangement, e.g. like the spatial correspondence arrangement 76, is provided that tracks a spatial relation of the X-ray imaging system 52 that provides the angiographic image data and an optical perfusion imaging system that provides the optical perfusion image data.

In an example, provided as an option, the data input 12 is configured to provide location data representing a relative spatial arrangement of an angiography imager when acquiring the angiographic image data and an optical measurement imager for acquiring the optical perfusion imaging. Further, the data processor 14 is configured to generate position adjustment instructions to arrange the optical measurement imager for providing measurement of the determined surface portion of the subject.

The term angiography imager relates to an imaging device or system capable of providing the angiographic image data.

The term optical measurement imager relates to an imaging device or system capable of providing the optical perfusion image data.

In an example, an angiography driven field-of-view selection for optical perfusion measurement is provided. If the optical perfusion measurement can only be applied to a limited field-of-view, the angiography imaging can be used for selecting the most relevant area to be measured in order to speed-up the clinical workflow. For this purpose, the angiography is acquired first. Having identified e.g. the clinically most relevant sole region in the angiography, e.g. a region with perfusion deficits in the depth where an optical measurement of the skin oxygenation is of clinical interest, the measured/known relative pose of the angiography and the optical measurement device is used to move the latter in space such that this most relevant sole region is fully covered. Such an angiography-driven field-of-view selection is provided, for example, if there are no visible signs of perfusion deficits on the skin surface, e.g. only slowly healing wounds.

In an example, the spatial correspondence arrangement 76 establishes the spatial correspondence between the two imaging modalities and transfers location information and hypotheses about the perfusion status from angiography to the optical surface domain for an advanced analysis of perfusion properties.

The spatial correspondence between the two imaging modalities is needed for registering both image domains or image spheres with each other.

In an example, angio images are provided together with information about the vascular types or segments. This allows a registration with the optical perfusion images. In another example, the angio images are provided without spatial information. A model is provided in order to assess spatial data such that the registration with the optical perfusion images is possible.

In an example, both optical perfusion measurements, e.g. hyper/multispectral iPPG with structured light, and angiography sequences are acquired from approximately orthogonal views. If e.g. cuff or other stimuli are applied, measurements are synchronized. In an example, in order to establish approximate spatial correspondences between the modalities, the imaging geometry, i.e. relative positioning of modalities and e.g. a foot, must be known or reconstructed: While a hardware-wise fixation, e.g. mounting the optical imaging system on the C-arm, might not be feasible, e.g. due to geometric constraints, the relative position and rotation of both imaging devices may be measured via sensors, e.g. (depth-) camera, Wi-Fi, radar. In one example, at least one ceiling mounted camera senses the position of reflective or active marker constellations attached to the gantry and the optical imaging system in cathlab coordinates. In another example, at least two angiography sequences are acquired from different views. As an alternative to sensors, pose-estimation algorithms are be applied to both the angiography and optically acquired images: For the angiography, a neural-network based matching of an articulated foot-model to the X-ray image is used. Utilizing the known change in the C-arm positioning, e.g. encoded in the DICOM metadata, the accuracy of the pose-estimation of angiography based on the acquired images can be enhanced. In an example, a pose of the optical imaging system is estimated using the acquired RGB or grey-level signal. This may be easily accomplished by using standard triangulation approaches with a second camera or projection device, e.g. light spatially structured in the plane orthogonal to its propagation direction.

In an example of a clinical workflow, DSA sequences are acquired prior to and after the lesion has been treated during an actual intervention. Both sequences, i.e. pre and post, show the macrovascular tree, which is perfused, i.e. contrast filled, at the time of imaging. The impact of the intervention on macrovascular perfusion can be assessed by comparing the two vessel trees. A perfused part of the vessel tree, and therefore also a change in perfusion, can be defined in one of the following ways:

Binary fashion: According to a certain intensity threshold on the contrast density, a vessel or part of the vessel is either perfused or not. Interventional impact is identified by vessel segments that are filled by a contrast density above the threshold after the intervention, but not before. The analysis can be done after reducing the DSA sequence to its MIP and segmenting the macrovascular tree. In an example, a segmentation mask is used to eliminate image parts which do not belong to the macrovascular tree before applying the threshold.

Time-density curve (TDC) parameter change: A softer criterium is applied, which analyses the contrast density curve of each pixel in the macrovasculature and extracts parameters from that curve, e.g. peak density, time-to-peak, plateau width and the like. In addition to newly appearing vessel segments, significant changes in one of the TDC parameters also qualify a vessel segment to be highlighted as impacted by the intervention.

In an example, the way the change in perfusion is measured provides an influence on how the (re-)prefusion hypothesis for the optical part is defined. While the thresholding approach above indicates that a perfusion change is expected optically which is maybe above some inter-repeated-measurement variability, the TDC approach is capable of also indicating the extent of expected surface perfusion change.

As an example, the analysis of a foot angiogram results in three outputs: the pre-interventionally perfused macrovasculature, the post-interventionally perfused macrovasculature and the part of the vessel tree that was affected by the intervention.

In an example, both angiographic X-ray projections (pre and post) are recorded from very similar angles as required by a stereo-imaging setup, their DSA-based MIPs (masked by the macrovascular segmentation) are warped onto each other to define the differences in the vascular trees. The warping, e.g. via thin plate splines or optical flow, matches coinciding parts of the vascular tree and lets the differing parts stand out.

In an example, locational and semantic information to these outputs is provided manually (by the user) or automatically, assigning vessel labels to vascular segments of the vessel tree. Vascular segments are defined and bordered by branching points/bifurcations and are assigned a vessel label, such as ATA1, ATA2, . . . , PTA1, PTA2, when identifiable from a reference anatomical atlas.

An automatic extraction of this information could start as an iterative approach seeded at the inlet vessels in the ankle region. From there the tracking travels down the vascular tree detecting and branching at encountered bifurcations.

Further, the outputs can be connected to a 3D reference model of the foot where they are linked to their corresponding feeding territories in the tissue. Here, an exact registration of the X-ray projected foot pose to the 3D mode is not necessary, because semantic information has been found in the 2D vessel tree, which can be used to identify correspondences to supply regions in the 3D model to which the optical scan is registered.

This can be done in several ways: By exploiting the vessel label and the angiosome model, a general population-wide model which indicates which parts of the foot are perfused by which vessel (ATA, PTA, peroneal, calcaneal artery . . . ); or by using an average vascular tree as part of the reference model, that was built in a data-driven manner from many contrasted 3D images of feet (CTA, MRA data) and that contains the vascular segment labels annotated in the DSA image. As an approximation, it is assumed (and computed via nearest neighbor search) that tissue regions closest to a particular vessel are also perfused by this vessel. Regions in the 3D model for which the perfusing vessel segment (present in the average reference tree in the foot model) is not observed/perfused in the angiogram, are labelled as not perfused or if a pre/post-interventional change was detected are labelled as impacted by the intervention; or diseased patients may exhibit a very individual vascular tree especially at the smaller caliber parts of the tree. This also includes collaterals and changes in the vasculature that have built up over time to compensate for the progressing disease. These aspects are less likely to be covered by the angiosome model or a standard reference vascular tree (if it does not come from a patient-specific 3D image). In this case, the connectivity (branching point locations) to the bigger caliber vessels (included in the angiosome model) can be exploited to estimate which tissue parts may be affected by a perfusion change in this individual part of the vascular tree or angiosome. Depending on the connectivity, this could give rise to a further narrowing down the affected sub-parts within the coarser angiosome parcellation.

By this procedure, it can be approximated which parts of the surface tissue are perfused to which extent and how it has been changing during the intervention.

Further, the hyper/multispectral iPPG surface scan can also be mapped to the reference foot model. By having both pieces of information (affected and relevant perfusion territory information from angiography & iPPG surface perfusion parameter map), the information can be overlaid on each other to evaluate the correlation between the interventional impact (macrovascular vessel tree information), its hypothesized impact on the perfusion change in the feeding territory (the mapping procedure in the foot model) as well as the actual impact on the surface tissue as observed by iPPG.

Expectations that the interventionalist has based on his or her treatment, can therefore be automatically compared with the real effect in the target region. The information can be automatically reported, and disagreements can be highlighted to the clinician. Going through this procedure several times, for example after the clinician has possibly extended the treatment, e.g. additional ballooning, opening another vessel etc., results in a documentation of incremental improvements. These provide insights into how changes in the surface tissue supply are related to macrovascular changes caused by the interventional effort, which can be used for a prognosis of what effects are still likely to be achieved via the intervention.

In an example of the method, a perfusion hypothesis is generated based on the comparing of the first image data and the second image data and the identification of the vascular region of interest for macrovascular flow. Further, as the identifier, instructions for optical perfusion imaging are generated for at least one of the group of quantifying and validating of the perfusion hypothesis.

In an example of the method, locational information and semantic information are provided for the vascular structure of at least one of the group of the first image data and the second image data. The semantic information is provided for the determining of the at least one tissue portion of interest for microvascular perfusion. The locational information is provided for the allocating of the surface portion.

In an example of the method, the angiographic image data and the subject are registered to a common spatial reference frame. The allocating of the surface portion comprises allocating a surface portion of the subject based on the common spatial reference frame.

In an example of the method, the angiographic image data comprises spatial anatomical information comprising a plurality of vessel segments. The identification of the vascular region of interest for macrovascular flow comprises identifying at least one of the plurality of vessel segments. The feeding information comprises segments of tissue portions that are assigned to vessel segments of the plurality of vessel segments as being supplied by these vessel segments.

In an example of the method, the feeding information comprises a feeding territory model. The angiographic image data is spatially registered to the feeding territory model. The feeding territory model is registered to the subject.

In an example of the method, the area of interest of the subject comprises the lower limbs. The at least one tissue portion of interest for microvascular perfusion comprises at least one foot region of the subject.

In an example of the method, optical perfusion image data is provided. The optical perfusion image data comprises at least one 2D optical perfusion image of the surface of the subject. An indicator is generated for the allocated surface portion; and the indicator is overlaid to the at least one 2D optical perfusion image to provide a location augmented optical perfusion image. The location augmented optical perfusion image is provided for a perfusion assessment in the image portion defined by the indicator.

In an example of the method, for overlaying the indicator, the at least one 2D optical perfusion image is registered with the angiographic image data.

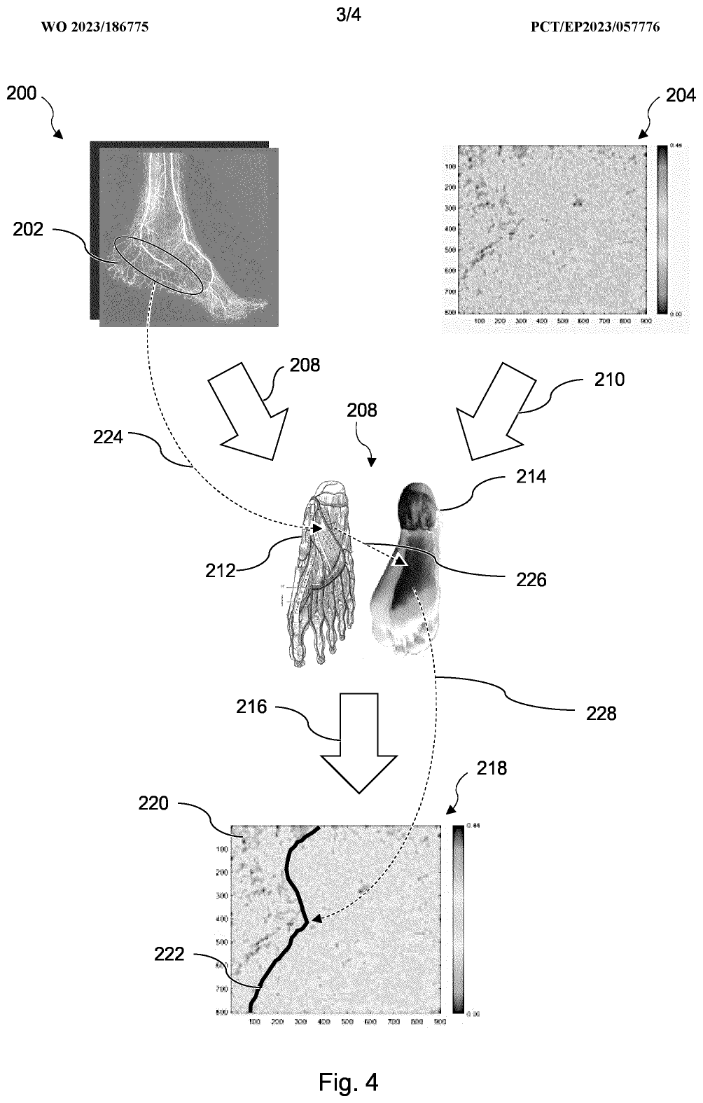

FIG. 4 illustrates an example of a workflow for peripheral perfusion imaging. FIG. 4 shows a schematic illustration of information flow for transferring expectations arising from macrovascular aspects as observed in the angiogram to the optically scanned skin surface area. The latter is assumed to be partially fed by the relevant macrovascular parts. The transfer is accomplished by using a vascular model that carries or approximates the information of which tissue is fed by which artery. Depending on the level of detail in the vascular tree, the feeding zones can be organized in a refinement hierarchy becoming finer with decreasing vessel calibers.

As indicated in FIG. 4, at least one pre interventional angiogram and at least one post interventional angiogram are provided, indicated with reference numeral 200. As an example, a perfusion dysfunction in lateral plantar artery is detected by comparing the pre and post angiograms, as indicated with reference numeral 202. Further, an optical perfusion image 204 is provided. In a first registration part 206, the pre/post angiograms 200 are registered to a vasculature/feeding territory model 208, e.g. an angiosome model. In a second registration part 210, the optical perfusion image 204 is registered to the vasculature/feeding territory model 208, e.g. the angiosome model. In FIG. 4, an anatomical foot model 212 is shown, together with a territory model 214. In a computational procedure, a surface portion for assessing microvascular perfusion in the at least one tissue portion of interest with optical perfusion imaging is determined. In an augmentation 216, data for displaying an augmented optical perfusion image 218 is provided which is then displayed showing optical perfusion image content 220 together with a surface portion identifier 222. Hence, by detecting the area of interest in the angiographic image data, this location is transferred to the foot model 212, as indicated by first hashed arrow 224, to determine the tissue portion of interest. This is then transferred to the territory model 214, as indicated by second hashed arrow 226, to determine the surface portion. From here, a further transformation is provided by allocating the surface portion on an outer surface of the subject, as indicated by third hashed arrow 228, to be able to present the surface portion identifier 222.

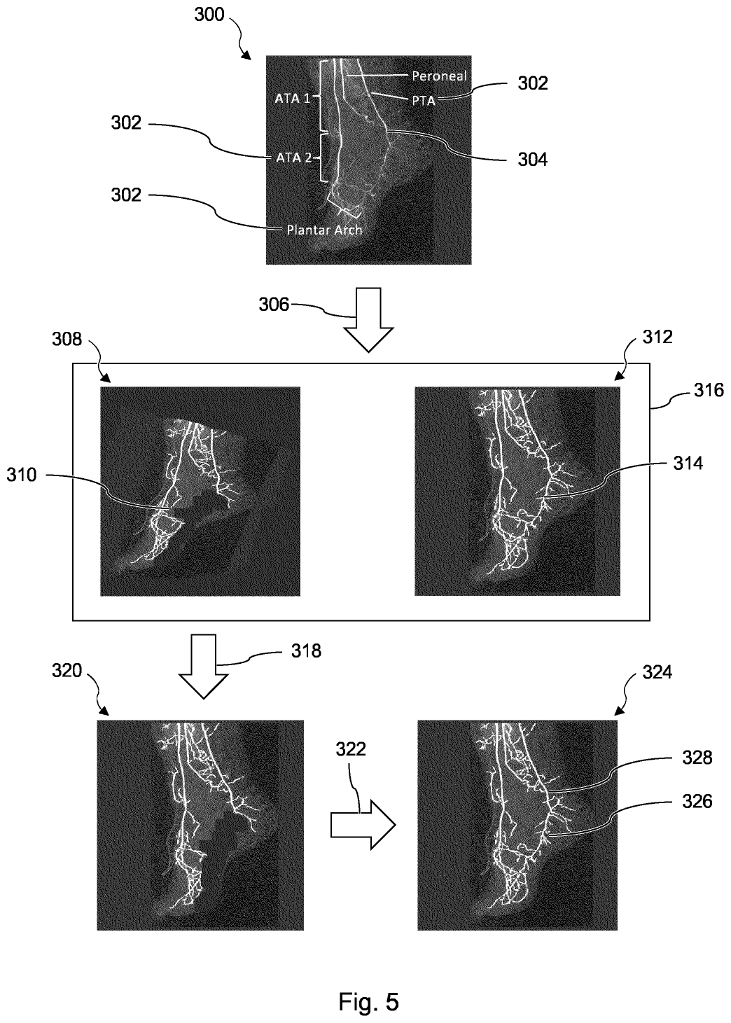

FIG. 5 illustrates a further example of a workflow for peripheral perfusion imaging. FIG. 5 shows extracting macrovascular information from pre-and post-interventional images. The macrovasculature is first segmented from the DSA MIP image and semantic labels are assigned to segments of the vessel tree. This procedure is done for the pre- and post-interventional image (top row). Slightly differing projection angles or foot poses between the two images are compensated by geometrically warping the coordinates of one into the other image (e.g. via thin-plate splines (TPS)). Using this match, the diff between the two vessel trees can be determined (bottom right in green). Finally, there are three pieces of information available for highlighting blood supply regions in the surface scan: pre-interventional macrovasculature, post-interventional macrovasculature, difference in macrovasculature between pre and post.

As indicated in FIG. 5, image data 300 of a digital subtraction angiography with and maximum intensity projection (DSA MIP) is provided. Semantic labels 302 are indicated for a macrovascular structure 304. A segmentation 306 results in a first macrovascular image 308 with a macrovascular structure 310, which relates to a pre interventional state, and a second macrovascular image 312 with a macrovascular structure 314, which relates to a post interventional state. A frame 316 indicates the option of a common data processing. Further, a warping procedure 318 results in a warped image 320. A difference is provided in a further procedure 322 resulting in a difference-image 324 in which differing parts 326 of the macrovascular structure 328 of the subject are highlighted.

The term “subject” may also be referred to as individual. The “subject” may further also be referred to as patient, although it is noted that this term does not indicate whether any illness or disease is actually present with the subject.

In an example, a computer program is provided that enables a data processor to carry out the method of the examples above.

In an example, a computer program or program element for controlling an apparatus according to one of the examples above is provided, which program or program element, when being executed by a processing unit, is adapted to perform the method steps of one of the method examples above, on an appropriate system.

In an example, a computer readable medium is having stored the program element of the previous example.

The computer program element might therefore be stored on a computer unit or be distributed over more than one computer units, which might also be part of an embodiment of the present disclosure. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the current disclosure.

Aspects of the current disclosure may be implemented in a computer program product, which may be a collection of computer program instructions downloadable from a communications network and/or stored on a computer readable storage device or medium which instructions may be executed by a computer such as for example a data processor as defined herein. The instructions of the present disclosure may be in any interpretable or executable code mechanism, including but not limited to scripts, interpretable programs, dynamic link libraries (DLLs) or Java classes. The instructions can be provided as complete executable programs, partial executable programs, as modifications to existing programs (e.g. updates) or extensions for existing programs (e.g. plugins). Moreover, parts of the processing of the present discloser may be distributed over multiple computers or processors. Thus the data processor may comprise multiple processors in data communication with each other to perform the functions as defined herein.

As discussed above, the processor, for instance a controller, is configured to implements any methods as defined herein. The controller can be implemented in numerous ways, with software and/or hardware, to perform the various functions required. A processor is one example of a controller which employs one or more microprocessors that may be programmed using software (e.g., microcode) to perform the required functions. A controller may however be implemented with or without employing a processor, and also may be implemented as a combination of dedicated hardware to perform some functions and a processor (e.g., one or more programmed microprocessors and associated circuitry) to perform other functions.

Examples of controller components that may be employed in various embodiments of the present disclosure include, but are not limited to, conventional microprocessors, application specific integrated circuits (ASICs), and field-programmable gate arrays (FPGAs).

This exemplary embodiment of the disclosure covers both, a computer program that right from the beginning uses the methods as disclosed herein and a computer program that by means of an update turns an existing program into a program that uses the methods as disclosed herein.

Further on, the computer program element might be able to provide all necessary steps to fulfil the procedure of an exemplary embodiment of the method as described above.

According to a further exemplary embodiment of the present disclosure, a computer readable medium, such as a CD-ROM, is presented wherein the computer readable medium has a computer program element stored on it which computer program element is described by the preceding section. A computer program may be stored and/or distributed on a suitable medium, such as an optical storage medium or a solid-state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present disclosure, a medium for making a computer program element available for downloading is provided, which computer program element is arranged to perform a method according to one of the previously described embodiments of the disclosure.

It has to be noted that embodiments of the current discloser are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the currently disclosed concepts have been illustrated and described in detail in the drawings and foregoing description, such illustration and description are to be considered illustrative or exemplary and not restrictive. The claims are not limited to the disclosed embodiments. Other variations to the disclosed embodiments can be understood and effected by those skilled in the art in practicing a disclosed concept, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A device for assisting monitoring of microvascular perfusion, comprising:
a data input;
a data processor; and
an output interface;
wherein the data input is configured to provide flow data comprising information about macrovascular blood flow in an area of interest of a subject;
wherein the data processor is configured to:
identify, based on the flow data, within the area of interest of the subject, a vascular region of interest for macrovascular flow;
determine at least one tissue portion of interest for microvascular perfusion, the determination based on the identified vascular region of interest and based on feeding information assigned to the identified vascular region of interest;
determine a surface portion for assessing microvascular perfusion with optical perfusion imaging; the surface portion being part of the at least one tissue portion of interest;
allocate the surface portion on an outer surface of the subject or organ thereof; and
wherein the output interface is configured to provide a surface portion identifier based on the allocated surface portion.

2. Device according to claim 1, wherein the flow data comprise or consist of angiographic image data comprising the information about macrovascular blood flow in an area of interest of the subject; wherein the angiographic image data comprise first image data relating to a first point in time, and second image data relating to a second point in time; and wherein the data processor is configured to:
compare the first image data and the second image data to identify, within the area of interest of the subject, the vascular region of interest for macrovascular flow.

3. Device according to claim 1, wherein the data processor is configured to generate a perfusion hypothesis based on the identification of the vascular region of interest for macrovascular flow; and
wherein the data processor is configured to generate, as the surface portion identifier, instructions for optical perfusion measurement for at least one of the group consisting of: quantifying and validating of the perfusion hypothesis.

4. Device according to claim 1, wherein the data input is configured to provide at least one of the group consisting of locational information and semantic information for the vascular structure;
wherein the data processor is configured to determine the at least one tissue portion of interest for microvascular perfusion using the semantic information; and/or
wherein the data processor is configured to allocate the surface portion using the locational information.

5. Device according to claim 4, wherein the data processor is configured to register the angiographic image data and the subject to a common spatial reference frame; and
wherein the data processor is configured to allocate the surface portion based on the common spatial reference frame.

6. Device according to claim 1, wherein the flow data, such as for example the angiographic image data, comprises spatial anatomical information comprising a plurality of vessel segments;
wherein, for the identification of the vascular region of interest for macrovascular flow, the data processor is configured to identify at least one of the plurality of vessel segments; and
wherein the feeding information comprises segments of tissue portions that are assigned to vessel segments of the plurality of vessel segments as being supplied and/or drained, preferably only supplied, by these vessel segments.

7. Device according to claim 1, wherein the feeding information comprises a feeding territory model;
wherein the data processor is configured to spatially register the angiographic image data to the feeding territory model; and
wherein the data processor is configured to register the feeding territory model to the subject.

8. Device according to claim 1, wherein the area of interest of the subject comprises the lower limbs; and
wherein the at least one tissue portion of interest for microvascular perfusion comprises at least one foot region of the subject.

9. Device according to claim 1,
wherein the data input is configured to provide optical perfusion image data comprises at least one 2D or 3D optical perfusion image of the surface of the subject or organ thereof;
wherein the data processor is configured to overlay or integrate the surface portion identifier to the at least one 2D or 3D optical perfusion image to provide location augmented 2D or 3D optical perfusion image data; and
wherein the output interface is configured to provide the location augmented optical perfusion image data.

10. Device according to claim 1, wherein, for overlaying or integrating the indicator, the data processor is configured to register the at least one 2D or 3D optical perfusion image with the angiographic image data.

11. Device according to claim 1, wherein the data input is configured to provide location data representing a relative spatial arrangement of an angiographic imaging system when acquiring the angiographic image data and an optical perfusion imaging system for acquiring the optical perfusion image; and
wherein the data processor is configured to generate position adjustment instructions to arrange the optical perfusion imaging system for providing measurement of the allocated surface portion of the subject.

12. A system for monitoring microvascular perfusion, comprising:
a device according to claim 1;
optionally, a source system for providing the flow data to the device; and
an optical perfusion measurement system for performing the optical perfusion measurement of the allocated surface portion based on the surface portion identifier.

13. System according to claim 12, wherein a spatial correspondence arrangement is provided that is configured to provide relative spatial correspondence data of the angiographic imaging system and the optical perfusion system.

14. A method comprising:
using a data input to provide to a data processor flow data comprising information about macrovascular blood flow in an area of interest of a subject;
using the data processor to:
identify, within the area of interest of the subject, a vascular region of interest for macrovascular flow;
determine at least one tissue portion of interest for microvascular perfusion, the determination being based on the identified vascular region of interest and based on feeding information assigned to the identified vascular region of interest;
determine, within the at least one tissue portion of interest, a surface portion for assessing microvascular perfusion with optical perfusion imaging,
allocate the surface portion on an outer surface of the subject or organ thereof; and
provide a surface portion identifier based on the surface portion.

15. Method according to claim 14, wherein the flow data comprise or consist of angiographic image data comprising the information about macrovascular blood flow in an area of interest of the subject; wherein the angiographic image data comprise first image data relating to a first point in time, and second image data relating to a second point in time; and wherein the method comprises comparing the first image data and the second image data to identify, within the area of interest of the subject, the vascular region of interest for macrovascular flow.

16. Method according to claim 14 wherein the method comprises:
generating a perfusion hypothesis based on the identified vascular region of interest for macrovascular flow; and
generating, as the surface portion identifier, instructions for optical perfusion measurement for at least one of the group consisting of: quantifying and validating of the perfusion hypothesis.

17. Method according to claim 1, wherein the feeding data comprises at least one of the group consisting of locational information and semantic information for the vascular structure; wherein the method comprises performing at least one of the group consisting of: determining the at least one tissue portion of interest for microvascular perfusion using the semantic information and allocating the surface portion using the locational information.

18. Method according to claim 14, wherein the flow data, such as for example the angiographic image data, comprises spatial anatomical information comprising a plurality of vessel segments; wherein the identification of the vascular region of interest for macrovascular flow comprises identification of at least one of the plurality of vessel segments; and wherein the feeding information comprises segments of tissue portions that are assigned to vessel segments of the plurality of vessel segments as being supplied and/or drained, preferably only supplied, by these vessel segments.

19. Method according to claim 14, comprising:
using a data input to provide optical perfusion image data comprising at least one 2D or 3D optical perfusion image of the surface of the subject or organ thereof;
using the data processor to:
overlay or integrate the surface portion identifier to the at least one 2D or 3D optical perfusion image to provide location augmented 2D or 3D optical perfusion image data; and
use the output interface to provide the location augmented optical perfusion image data;
optionally, use a user interface to provide the location augmented optical perfusion image to a user.

20. A computer program downloadable from a communications network or stored on a computer readable medium, the computer program comprising computer readable code which when run on a data processor or computer as defined herein causes a device as claimed herein or a system as claimed herein to carry out the steps of method 14.
